(19) United States
(12) Patent Application Publication
(10) Pub. No.: US 2012/0156717 A1
(43) Pub. Date: Jun. 21, 2012

(54) BIOFUEL FROM RECOMBINANT OLEAGINOUS ALGAE USING SUGAR CARBON SOURCES

(75) Inventors: F.C. Thomas Allnutt, Glenwood, MD (US); Bradley Lynn Postier, St. Charles, MO (US); Richard T. Sayre, Webster Groves, MO (US); Daniel A. Coury, St. Louis, MO (US)

(73) Assignee: Phycal, Inc., Highland Heights, OH (US)

(21) Appl. No.: 13/392,950

(22) PCT Filed: Aug. 30, 2010
(86) PCT No.: PCT/US10/47091

§ 371 Date: Feb. 28, 2012

Related U.S. Application Data
(60) Provisional application No. 61/238,072, filed on Aug. 28, 2009.

(51) Int. Cl. C12P 1/00; C12P 9/00; C12P 33/00; C12N 1/13; C12P 7/64

(52) U.S. Cl. 435/52; 435/257.2; 435/41; 435/134; 435/131

(57) ABSTRACT

Recombinant oleaginous alga that include one or more heterologous genes that increase the ability of the alga to use one or more natural saccharides such as cellulosic or hemicellulosic sugars for algal growth are described. The recombinant oleaginous algae are transformed to include one or more genes expressing sugar metabolizing enzymes or sugar transporting proteins, along with suitable control elements. Use of natural saccharides as a carbon source can allow the algae to produce biofuel precursors in a relatively efficient manner. Processes for preparing the alga, growing the alga, and extracting the biofuel precursors from the alga are also described.

### CROSS-REFERENCE TO RELATED APPLICATIONS

This application claims priority to U.S. Provisional Patent Application No. 61/238,072, filed Aug. 28, 2009, the entirety of which is incorporated herein by reference.

### BACKGROUND

Recent research indicates that microalgal biofuel systems can contribute substantially to domestic energy production in a sustainable and potentially carbon neutral manner. Chisti Y, Biotech Adv 25: 294-306 (2007). Microalgae can utilize feedstock materials that do not directly compete with food production, are 10-30 times more productive per hectare than terrestrial biofuel systems, can be harvested daily due to rapid doubling times, and can capture industrial CO₂ emissions. To be competitive with petroleum-based fuels, however, microalgal biomass production and oil harvesting costs must be substantially reduced from current estimates of about $30/gallon.

Oils, including polar membrane lipids and triacylglycerols (TAGs), typically make up 5-15% of the total biomass of algae. Oils can accumulate, however, up to 60% (w/w) of the total biomass in many algae when they are grown under conditions where the capacity to produce or metabolize reduced carbon exceeds the energy and carbon demands for growth. One way to encourage the accumulation of high levels of oil is to use heterotrophic growth, i.e., the use of an organic carbon source rather than sunlight and CO₂, to provide an energy source for the algae. Heterotrophic growth enhances both cell division and sequestration of reduced carbon as oil.

As a result of its abundance and renewable nature, biomass represents an excellent potential carbon source for producing affordable and environmentally sound biofuels to replace fossil fuels. Many biomass feedstocks can be used for the production of biofuel, such as lignocellulosic biomass (e.g., jatropha, switchgrass, and poplar), algae (e.g., macroalgae and microalgae), and oil crops (e.g., soybean, palm oil and sunflower). These biomass feedstocks have the advantage of being relatively inexpensive, but are relatively complex chemically and typically require deconstruction to obtain the useful biomass components (e.g., monosaccharides, fatty acids, etc.) that can be readily converted into biofuels. Starch (e.g., derived from corn, wheat, barley, etc.) and sugar crops (e.g., derived from cane, beet, etc.) are feedstocks that are easier to process, but are more expensive and compete with food production.

Lignocellulosic biomass includes a large amount of cellulose, which is a polymeric form of glucose, and hemicellulose, which includes sugars such as xylose, mannose, galactose, rhamnose, and arabinose. However, many algae do not include the transporter proteins and/or sugar metabolizing enzymes necessary to utilize these sugars derived from lignocellulosic biomass. Selective growth has been used to encourage Chlorella algae to grow using pentose sugars. See Hawkins, R., Curr. Microb., 38, p. 360-363 (1999). However, selective growth is an imprecise and unreliable method for modifying algae to be able to utilize alternative carbon sources. Accordingly, the need remains for algae that are able to convert natural saccharides, such as cellulosic and hemicellulosic sugars, into biofuel precursors, such as oils and lipids.

### SUMMARY OF THE INVENTION

The need for algae able to convert natural saccharides into biofuel precursors and other unmet needs of the prior art is met by exemplary compositions and methods described herein.

Cellulose and hemicellulose are estimated to be the most abundant organic molecules on earth. The efficient and complete conversion of cellulose or hemicellulose into biofuel precursors through the heterotrophic growth of algae provides significant advantages, even over fermentation of the same substrates to ethanol or butanol. Biofuel derived from the process has a higher energy density, is compatible with existing transportation engines and distribution systems, is non-toxic, and is not limited by final concentration of endproduct in the reactor vessel, such as occurs for ethanol and butanol. As such biofuel precursors, derived from algae produced phototrophically or heterotrophically are an excellent replacement fuel for petroleum based diesel and jet fuel. Since oleaginous algae are highly efficient (up to 90% energy conversion efficiency) at converting reduced carbon into oil, the recombinant oleaginous algae described herein can efficiently convert natural saccharides such as cellulose into a liquid, high-energy density fuel.

In one aspect, the present invention provides a recombinant oleaginous alga that includes one or more heterologous genes that increase the ability of the alga to use one or more natural saccharides for algal growth. In some embodiments, the natural saccharide is a cellulosic or hemicellulosic sugar, or a monosaccharide derived therefrom. In further embodiments, the one or more heterologous genes express one or more sugar metabolizing enzymes that are lacking in a wild-type form of the oleaginous alga. In a further embodiment, the one or more heterologous genes express one or more sugar transporters that are lacking in a wild-type form of the oleaginous alga. In additional embodiments, the recombinant algae also includes a nucleotide sequence expressing an RNAi construct or an expression vector that alters the metabolism of the algae to produce a larger amount of biofuel precursors such as fatty acids.

In another aspect, the present invention provides a method of making biofuel precursors that includes growing an algal culture including a recombinant oleaginous alga of any one of the preceding claims using a natural saccharide as a primary carbon source, and extracting one or more biofuel precursors from the algal culture. In some embodiments, the natural saccharide is a cellulosic or hemicellulosic sugar, or a monosaccharide derived therefrom. In further embodiments, the biofuel precursors are extracted by mixing a portion of the algal culture with an oil-extracting solvent to obtain a solvent-algae mixture, partitioning the solvent-algae mixture to obtain a solvent-biofuel precursor fraction and viable extracted algae, and returning the viable extracted algae to the algal culture.

### BRIEF DESCRIPTION OF THE FIGURES

The present invention may be more readily understood by reference to the following drawings wherein:

FIG. 1 provides a construct for containing ADP glucose pyrophosphorylase RNAi that allows one to block starch synthesis.

FIG. 2 provides two graphs showing the ability of various natural saccharides to support growth (FIG. 2A) or oil production (FIG. 2B) in Chlorella protothecoides.

FIG. 3 provides a scheme showing the enzymes involved in rhamnose metabolism.

FIG. 4 provides a schematic representation of a three gene operon from Clostridium that encodes the genes required for metabolism of rhamnose.

FIG. 5 provides a schematic illustration of the oxidation/reduction (redox) catabolic pathway for arabinose in fungi.

FIG. 6 illustrates a generalized pathway for metabolizing free galactose in the cell, showing the galactose metabolic enzymes involved.

To illustrate the invention, several embodiments of the invention will now be described in more detail. Skilled artisans will recognize the embodiments provided herein have many useful alternatives that fall within the scope of the invention.

### DETAILED DESCRIPTION OF THE INVENTION

The present invention provides methods and recombinant algae useful for the production of biofuel precursors. In particular, the invention provides recombinant algae with an increased capacity to use natural saccharides, such as cellulosic and hemicellulosic sugars, as a carbon source for heterotrophic growth.

### Definitions

Unless otherwise defined, all technical and scientific terms used herein have the same meaning as commonly understood by one of ordinary skill in the art to which this invention pertains. In case of conflict, the present specification will control.

The terminology as set forth herein is for description of the embodiments only and should not be construed as limiting of the invention as a whole. Unless otherwise specified, “a,” “an,” “the,” and “at least one” are used interchangeably. Furthermore, as used in the description of the invention and the appended claims, the singular forms “a,” “an,” and “the” are inclusive of their plural forms, unless contraindicated by the context surrounding such. The singular “alga” is likewise intended to be inclusive of the plural “algae.”

As used herein, the term “gene” refers to a nucleic acid that encodes a stretch of DNA that codes for a type of protein or for an RNA chain that has a function in the organism. The term “gene” refers broadly to any segment of DNA associated with a biological function. As such, the term “gene” encompasses sequences including but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation from one or more existing sequences.

The term “express” in the context of a gene generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence the biological activity of a gene or gene product. These processes include, but are not limited to RNA synthesis, processing, and transport, as well as polypeptide synthesis, transport, and post-translational modification of polypeptides. Additionally, processes that affect protein-protein interactions within the cell can also affect gene expression as defined herein.

The terms “comprising” and variations thereof do not have a limiting meaning where these terms appear in the description and claims.

Also herein, the recitations of numerical ranges by endpoints include all numbers subsumed within that range (e.g., 1 to 5 includes 1, 1.5, 2, 2.75, 3, 3.80, 4, 5, etc.).

The phrase “recombinant algae,” as used herein, refers to algae whose genetic material has been altered using genetic engineering techniques so that it is no longer a “wild type” organism. An example of recombinant algae are transgenic algae that possess one or more genes that have been transferred to the algae from a different species Another example is an alga wherein endogenous genes have been rearranged such that they are in a different and advantageous arrangement yet no foreign DNA remains in the modified cell.

The term “wild-type,” as used herein, refers to the phenotype of the typical form of a species as it occurs in nature. Alternatively, the term can be used to refer to the typical, natural form of a species when viewed as a whole. It is appreciated that most or all gene loci exist in a variety of allelic forms which vary in frequency throughout the geographic range of a species, and that a uniform wild type does not exist. However, where ambiguity such as this exists, the most prevalent allele; i.e., the one with the highest gene frequency, is the one deemed to be the wild-type.

The term “transfection” refers to the introduction of a nucleic acid, e.g., an expression vector, into a recipient cell, which in certain instances involves nucleic acid-mediated gene transfer. The term “transformation” refers to a process in which a cell's genotype is changed as a result of the cellular uptake of heterologous nucleic acid. For example, a transformed cell can express a sugar metabolizing enzyme or a sugar transporter that is not present in the typical genotype of the alga cell.

The transformation of a cell with a heterologous nucleic acid (for example, an expression vector) can be characterized as transient or stable. As used herein, the term “stable” refers to a state of persistence that is of a longer duration than that which would be understood in the art as “transient”. These terms can be used both in the context of the transformation of cells (for example, a stable transformation), or for the expression of a transgene (for example, the stable expression of a gene encoding a sugar metabolizing enzyme) in a transgenic cell. In some embodiments, a stable transformation results in the incorporation of the heterologous nucleic acid molecule (for example, an expression vector) into the genome of the transformed cell. As a result, when the cell divides, the vector DNA is replicated along with plant genome so that progeny cells also contain the heterologous DNA in their genomes. Transformation of plastid or other self replicating organelle based nucleic acids (e.g., chloroplast or mitochondrial DNA) is also contemplated under this definition.

The term “vector” refers to a nucleic acid capable of transporting another nucleic acid to which it has been linked. One type of vector that can be used in accord with the presently disclosed subject matter is an Escherichia Coli vector, i.e., a nucleic acid capable of integrating the nucleic acid sequence of interest into the host cell (for example, an algal cell) genome. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of plasmids. In the present specification, “plasmid” and “vector” are used interchangeably as the plasmid is the most commonly used form of vector. However, the presently disclosed subject matter is intended to include such other foams of expression vectors which serve equivalent functions (e.g., cosmids or bacmids) or and which become known in the art subsequently hereto.

The term “expression vector” as used herein refers to a DNA sequence capable of directing expression of a particular nucleotide sequence in an appropriate host cell. Expression vectors can contain a variety of control sequences (e.g., promoters and terminators), structural genes (e.g., genes of interest), and nucleic acid sequences that serve other functions as well. The construct comprising the nucleotide sequence of interest can be chimeric. The nucleotide sequence of interest, including any additional sequences designed to effect proper expression of the nucleotide sequences, can also be referred to as an “expression cassette”.

The terms “heterologous nucleic acid sequence”, or “heterologous gene”, as used herein, each refer to a sequence that originates from a source foreign to an intended host cell or, if from the same source, is modified from its original form. Thus, a heterologous gene in a cell contemplates the possibility that a gene can be endogenous to the particular cell but has been modified, for example by mutagenesis or by isolation from native transcriptional regulatory sequences. The terms also include non-naturally occurring multiple copies of a naturally occurring nucleotide sequence. Thus, the terms refer to a DNA segment that is foreign or heterologous to the cell, as well as genes that are homologous to the cell but in a position within the host cell nucleic acid wherein the element is not ordinarily found.

The terms “promoter” or “promoter region” each refer to a nucleotide sequence within a gene that is positioned 5′ to a coding sequence and functions to direct transcription of the coding sequence. The promoter region comprises a transcriptional start site, and can additionally include one or more transcriptional regulatory elements. Different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell.

The term “operatively linked”, when describing the relationship between two nucleic acid regions, refers to a juxtaposition wherein the regions are in a relationship permitting them to function in their intended manner. For example, a control sequence “operatively linked” to a coding sequence can be ligated in such a way that expression of the coding sequence is achieved under conditions compatible with the control sequences, such as when the appropriate molecules (e.g., promoters and/or terminators) are bound to the control or regulatory sequence(s). Thus, in some embodiments, the phrase “operatively linked” refers to a promoter connected to a coding sequence in such a way that the transcription of that coding sequence is controlled and regulated by that promoter. Techniques for operatively linking a promoter to a coding sequence are well known in the art; the precise orientation and location relative to a coding sequence of interest is dependent, inter alia, upon the specific nature of the promoter.

Embodiments of the invention are directed at the conversion of natural saccharides to hydrocarbons such as, but not limited to, mono-, di- and triacylglycerides. The purpose of the invention is to allow algae to convert natural saccharides as a feedstock into biofuel precursors at high efficiency. Algal strains will be genetically engineered to express genes important to the uptake and metabolism of natural saccharides such as cellulosic or hemicellulosic sugars to provide simpler fixed carbon compounds that can be converted to oil. Such an approach can also be used for production of alternative fuels such as ethanol, butanol, methanol, isobutanol, mixed alkanes, and the like.

In one aspect, the present invention provides a recombinant oleaginous alga that includes one or more heterologous genes that increase the ability of the alga to use one or more natural saccharides for algal growth. In another aspect, the invention provides a recombinant alga that includes one or more heterologous genes that increase the ability of the alga to use one or more cellulosic or hemicellulosic sugar for algal growth. The wild-type of the oleaginous alga may lack genes that express sugar metabolizing enzymes, or it may already have a capacity for metabolizing sugar as a carbon source that is merely being increased by transformation of the alga. Alternately, or in addition, the wild-type alga can include the enzymes for metabolizing a sugar but lack a transporter for uptake of the sugar into the cell.

An oleaginous alga, as referred to herein, is an algal species that can, under known conditions, accumulate a significant portion of its biomass as biofuel precursors (i.e., an oil or lipid). For example, embodiments of oleaginous algae are algae species that are capable of accumulating at least 10%, at least 20%, at least 30%, at least 40%, or at least 50% of their biomass as lipid. Suitable oleaginous algal species can be found in the Bacillariophyceae, Chlorophyceae, Cyanophyceae, Xanthophyceae, Chrysophyceae, Chlorella, Crypthecodinium, Schizocytrium, Nannochloropsis, Ulkenia, Dunaliella, Cyclotella, Navicula, Nitzschia, Cyclotella, Phaeodactylum, and Thraustochytrid classes and genera. A preferred genus of oleaginous algae is Chlorella, which includes numerous species capable of accumulating up to about 55% of their total biomass as lipids. See for example Miao & Wu, Journal of Biotechnology, 110, p. 85-93 (2004). Chlorella species include Chlorella kessleri (a.k.a. Parachlorella kessleri), Chlorella vulgaris, Chlorella protothecoides, and Chlorella sorokiniana. It is also noted that the nomenclature used to identify algae is subject to change, and that the algae genera and species recited are intended to cover the organisms referred to by those names at the time of filing, and should encompass the same organisms under subsequently revised nomenclature.

In some embodiments of the invention, the oleaginous alga is an alga that is not typically considered to be oleaginous in its natural, wild-type form, but which has been modified to be an oleaginous alga. This modification can occur as a result of selective growth, or through genetic transformation. For example, a non-oleaginous wild-type alga may have desirable characteristics such as a high growth rate that make it attractive for use in biofuel production. A wide variety of algae genera may be used in this way. Examples of alga that are not necessarily considered to be oleaginous in their wild-type, but may be modified to be oleaginous and are for other reasons attractive candidates for biofuel production include Chlamydomonas, Volvox, Galdiera, Coccomyxa, Ostreococcus, Hematococcus, Micromonas, Spirulina, Synechocystis, Synecoccocus, Anabaena, and Nostoc genera. Galdiera, for example, is an attractive candidate for modification for biofuel precursor production as it can metabolize over 50 sugars, sugar alcohols, amino acids, and other biosynthetic intermediates.

### Incorporation of Sugar Metabolizing Enzymes in Recombinant Oleaginous Algae

In order to make use of a more diverse range of biofuel feedstocks, the recombinant oleaginous algae of the present invention have been modified to include one or more heterologous genes that increase the ability of the alga to use one or more natural saccharides. In one embodiment, one or more heterologous genes express one or more sugar metabolizing enzymes that are lacking in a wild-type faun of the oleaginous algae. The oleaginous algae can be modified to include a single gene expressing a sugar metabolizing enzyme, or can be modified to include a plurality of genes expressing sugar metabolizing enzymes. Further, the plurality of genes can be directed to a single enzyme, or they can be directed to different types of enzymes. For example, the plurality of genes can be directed to a number of sugar metabolizing enzymes that operate together to metabolize a particular sugar substrate. If a plurality of genes is included, the genes can be included in the recombinant oleaginous alga as an operon, or alternatively they can be included as sequential genes with distinct control elements.

The algae of the invention are modified to increase their ability to use natural saccharides. Saccharides, also known as carbohydrates, are organic compounds with the general formula Cₘ(H₂O)ₙ, and structures that are well known by those skilled in the art. Natural saccharides are sugars that are commonly produced by biological processes in nature, in contrast with artificial sugars that are only prepared synthetically. Natural saccharides include a variety of different sizes of saccharides, and include monosaccharides, disaccharides, oligosaccharides, and polysaccharides. Monosaccharides include both hexose and pentose sugars. Examples of natural polysaccharides include cellulosic and hemicellulosic sugars.

Another aspect of the invention provides recombinant oleaginous algae that have been modified to increase the ability to use one or more cellulosic or hemicellulosic sugar(s). Cellulosic and hemicellulosic sugars, as described herein, encompass both monosaccharide and polymeric forms of cellulosic and hemicellulosic sugars, as well as various intermediate forms, such as disaccharides. Cellulose is composed entirely of D-glucose. Hemicellulose, on the other hand, contains many different sugar monomers. In addition to glucose, the sugar monomers in hemicellulose can include xylose, mannose, fucose, galactose, rhamnose, and arabinose. Hemicelluloses can include most of the D-pentose sugars, and occasionally small amounts of L-sugars as well. Xylose is always the sugar monomer present in the largest amount, but mannuronic acid and galacturonic acid also tend to be present. Cellulose is a straight chain polymer of cellulosic sugar, whereas hemicellulose is a branched chain polymer of hemicellulosic sugars.

Accordingly, in some embodiments of the invention, the recombinant oleaginous alga includes one or more genes expressing sugar metabolizing enzymes that are selected from enzymes capable of metabolizing the sugars glucose, rhamnose, arabinose, galactose, mannose, fucose, and xylose. The enzymes capable of metabolizing cellulosic and hemicellulosic sugars such as these are known to those skilled in the art. By “capable of metabolizing,” it is meant that the enzymes take part in the metabolic pathway involved in catabolism of the sugar. The number of enzymes that are used to transform the oleaginous alga can vary depending on the enzymes that are already present in the wild-type form of the alga. However, sufficient enzymes should be provided to carry out the catabolism of the sugar of interest. In some embodiments of the invention, the sugar metabolizing enzymes are obtained from other plants, other microorganisms such as fungi or bacteria, or other algae species (e.g., Galdieria species)

For example, the recombinant oleaginous alga may be modified to include genes expressing enzymes that metabolize the sugar arabinose. If the sugar substrate is arabinose, the sugar metabolizing enzymes provided in the recombinant oleaginous alga can include one or more of 1-arabinose reductase, 1-arabitol 4-dehydrogenase, and 1-xylulose reductase. The genes for these arabinose metabolizing enzymes are absent in many algal genomes and are required for arabinose metabolism. See Fonseca et al., FEBS J., 274, 3589-600 ((2007). These genes can be cloned with suitable algal-specific gene promoter/terminator combinations and cloned into expression vectors (e.g., E. coli expression vectors) for use in manipulation of algal metabolism.

The recombinant oleaginous alga can also be modified to include genes expressing enzymes that metabolize the sugar rhamnose. In this case, the sugar metabolizing enzymes can include one or more of L-rhamnose isomerase, rhamnulokinase, and rhamnulose-1-phosphate aldolase. The genes encoding the proteins responsible for metabolizing rhamnose can be found in Clostridium and can be provided together in the rhaABD operon (L-rhamnose isomerase, rhamnulokinase, and rhamnulose-1-phosphate aldolase). Alternately, the genes encoding the enzymes can be sequentially cloned with suitable control elements (e.g., promoters and/or terminators) for each structural gene. See Moralejo et al., J Bacteriol. 175, p. 5585-94 (1993).

The recombinant oleaginous alga can also be modified to include genes expressing enzymes that metabolize the sugar galactose. Sugar metabolizing enzymes that confer the ability to metabolize galactose include one or more of UDP glucose-hexose-1-phosphate uridylyltranserase and UDP glucose-hexose-1-phosphate uridylyltransferase. The genes expressing UDP glucose-hexose-1-phosphate uridylyltransferase and UDP glucose-hexose-l-phosphate uridylyltransferase are often missing in many algal genomes, and are required for galactose metabolism. These genes can be obtained from Saccharomyces cerevisiae or Trichoderma reesei, for example, and can be cloned with the algal-specific appropriate gene promoter/terminator combinations and cloned into expression vectors (e.g., E. coli expression vectors). For a description on obtaining these genes from Trichoderma reesei, see Martinez et al., Nat Biotech. 26, 553-560 (2008). The targeted gene cassette is cloned into an appropriate vector for amplification and is introduced into the algae of interest.

The recombinant oleaginous alga can also be modified to include genes expressing enzymes that metabolize the sugar xylose. To provide the capacity to metabolize xylose, one or more of the sugar metabolizing enzymes xylose isomerase, xylose reductase, xylitol dehydrogenase, and xylulokinase should be included. One or more of these enzymes are often missing in many algal genomes and are required for xylose metabolism. See Hawkins R., Current Microbiology, 38, p. 360-363 (1999). These genes can be obtained from Saccharomyces cerevisiae, Pichia stipitis, or Trichoderma reesei, for example, and can be cloned with the algal-specific appropriate gene promoter/terminator combinations and cloned into an expression vector (e.g., E. coli expression vectors). See Jeffries et al., Nat Biotech. 25, 319-326 (2007) and Martinez et al., Nat Biotech. 26, 553-560 (2008). The targeted gene cassette can be cloned into an appropriate vector for amplification and then introduced into the algae of interest.

The recombinant oleaginous alga can also be modified to include genes expressing enzymes that metabolize the sugar mannose. In this case, the sugar metabolizing enzymes are chosen from one or more of hexokinase, mannokinase, ATP-dependent fructokinase, and mannose-6-phosphate isomerase. The targeted gene cassette can be cloned into an appropriate vector for amplification (e.g., an E. coli expression vector) and then introduced into the algae of interest.

Cellulose and hemicellulose are polymeric natural saccharides that are made up of either cellulosic or hemiceullulosic monosaccharide subunits, respectively. The polymeric form of the sugars may require additional processing to convert at least a portion of the polysaccharide to smaller molecules, such as monosaccharides, before these sugars can be used as a carbon source. These monosaccharides are thereby “derived from” the cellulosic or hemicellulosic sugars. This is species specific and can be modified using the techniques described herein. This processing can be carried out in at least two different ways. For example, the cellulose and/or hemicellulose can be processed to form monosaccharides before being provided to the recombinant alga. Alternately, or in addition, the recombinant alga can include or be modified to include enzymes capable of digesting the cellulose and/or hemicellulose to form glucose or hemicellulosic monosaccharides (e.g., xylose, mannose, galactose, rhamnose, and arabinose). Examples of enzymes capable of degrading cellulose and hemicellulose include endo-cellulase, exo-cellulase, cellobiase, oxidative cellulases, xylanase, mannanase, β-glucosidase, endoglucosidase and cellulose phosphorylase.

Recombinant oleaginous algae can be modified to include enzymes capable of digesting cellulose or hemicellulose. However, for these enzymes to be effective they must be secreted into the environment where they cleave the cellulose and/or hemicellulose. This requires the digestive enzyme to include a signal sequence (i.e., a leader sequence) to facilitate export to the endoplasmic reticulum. The signal sequence is typically the first 20 or so amino acids at the N-terminal end of an expressed protein, and are typically highly hydrophobic. The signal sequence is cleaved from the protein by a signal peptidase once the desired transport has occurred. The nucleotide sequence encoding the signal sequence should be included at the 5′ end the gene for the digestive enzyme in the expression cassette used to modify the oleaginous algae to be able to cleave cellulose and/or hemicellulose. Suitable signal sequences are well known to those skilled in the art.

In addition to the natural saccharides found in cellulose or hemicellulose, the recombinant oleaginous alga of the invention can also be modified to increase their ability to use other natural saccharides as carbon sources. For example, the alga can be modified to provide the capacity to use sucrose, which is a disaccharide composed of glucose and fructose. Sucrose is a very common carbohydrate and can be obtained from a number of sources such as sweet sorghum. In order to provide the capability to metabolize sucrose, it is generally sufficient to provide the alga with the gene expressing an invertase, such as the cell wall invertase that is found in many higher plants. The role of Arabidopsis cell-wall invertases in sucrose metabolism is further described by Sherson et al., J. Exp. Botany, 54, p. 525-531 (2003).

### Incorporation of Sugar Transporting Proteins in Recombinant Oleaginous Algae

Algae can use sugar transporters to uptake natural saccharides from their environment so that these natural saccharides can be used as a carbon source by the algae. Accordingly, in an additional aspect of the invention, the recombinant oleaginous alga is transformed to include one or more genes expressing one or more sugar transporter proteins. Use of a transporter gene construct will facilitate the ability of the alga to transport the saccharide into the cell so that it can metabolize natural saccharides for use as a carbon source to produce biofuel precursors. The recombinant alga can be modified to express higher levels of sugar transport proteins than are found in the wild-type form of the alga. Alternately, or in addition, the alga can be modified to include heterologous genes expressing sugar transport proteins that are lacking in a wild-type oleaginous alga in order to provide the ability to take up more or different types of natural saccharides.

In some instances, it may be preferable to transfect the recombinant oleaginous alga with both sugar metabolizing enzymes and sugar transport proteins. For example, if the wild-type form of the oleaginous alga lacks the capacity to use particular natural saccharides as a carbon source, it may lack both sugar transport proteins and sugar metabolizing enzymes, and therefore need to be provided with both. If genes for both a sugar metabolizing enzyme and a sugar transporting protein are provided, it may be preferable to coordinate the type of sugar transporting protein being provided with the type of sugar metabolizing enzymes being expressed. In particular, if the oleaginous alga has been transformed to include one or more heterologous genes expressing one or more sugar metabolizing enzymes that are lacking in a wild-type form of the oleaginous alga, it may be preferable to also include genes expressing sugar transporters that can uptake the type of sugar or sugars that the recombinant oleaginous alga have been modified to be able to catabolize. However, it should be noted that it will not always be necessary to include a corresponding sugar transport protein because many sugar transport proteins have the ability to uptake a variety of different sugars. As a result, some embodiments of the invention may include sugar metabolizing enzymes for a plurality of different natural saccharides, but express only a single type of sugar transport protein that is capable of uptaking all of the different natural saccharides that the oleaginous alga is capable of metabolizing.

A number of different sugar transport proteins are known to be used by algae and higher plants. For example, the structure, function and physiology of a number of monosaccharide transporters are described by Büttner et al., Biochim Biophys Acta., 1465, pgs. 263-74 (2000). One sugar transporter that enables the uptake of a variety of different monosaccharides is the STP1 transporter. The STP1 transporter has been shown to be able to uptake mannose, galactose, xylose, glucose, fucose, fructose, and arabinose, with a typical preference for the D-form of the sugar. A number of other STP transporters (STP2-STP14) exist and transport a number of different sugar substrates. One source for STP transporter genes is the plant Arabidopsis.

Other known sugar transporters include HUP1, HUP2, and HUP3, which are naturally found in the green algae Chlorella (e.g., Chlorella kessleri). Like the STP transporters, the hexose uptake transporters are also able to uptake a variety of different natural saccharides As demonstrated by Stadler et al., the hexose uptake transporters are capable of uptaking a variety of different monosaccharides, including glucose, fructose, galactose, mannose, and xylose. Stadler et al., Plant Physiol. 107, p. 33-4 (1995). It has been shown that there is significant sequence homology between many plant monosaccharide transporters.

A variety of sugar transporters have been identified in the red algae Galdieria sulphuraria. Barbier et al., Plant Physiol. 137, 460-474 (2005) and Weber APM, Curr Opin Plant Biol 7, 247-253 (2004). These proton symporters are capable of shuttling glucose, galactose, fructose, and arabinose into the cytoplasm. These include the putative sucrose transporters also identified in the same work.

In addition to one or more genes expressing a monosacharide transporter, it may be preferable to include genes expressing sugar transporters capable of uptaking disaccharides such as the disaccharide sucrose. The structure and function of a number of sucrose transporters in plants is reviewed by Lemoine, R., Biochim Biophys Acta., 1465, 246-62 (2000). Examples of sucrose transport proteins include SUC1, SUC2 and SUT1-SUT4. For wild-type algae that lack the capacity to metabolize sucrose, it may be preferable to transform the algae to express both sucrose transport proteins as well as an invertase in order to uptake and then cleave the sucrose into glucose and fructose. The glucose and fructose are then further metabolized by the algae to produce biofuel precursors. For example, fructose can be converted by a fructose kinase into fructose-6-phosphate, which then is metabolized in glycolysis.

### Preparation of Recombinant Oleaginous Algae

The recombinant oleaginous alga of the invention can be made by transforming an alga cell with a nucleic acid fragment including a nucleotide sequence encoding a sugar metabolizing enzyme and/or a sugar transporter protein. Methods of transforming algal cells are well known in the art. For example, several Chlorella species have been transformed. Chen et al., Current Genetics 39: 365-370 (2001); Dawson et al., Current Microbiol., 35, 356-62 (1997); Chow and Tung, Plant Cell Reports 18. 778-780 (1999), El-Sheekh, Biologia Plantarum 42: 209-216 (1999); Grossman, A., Pl. Physiol. 137, 410-437 (2005). The technologies of electroporation, particle bombardment and glass beads used to transform Chlorella spp. are essentially identical to those used for Chlamydomonas transformation, and can be expected to be effective for a wide variety of oleaginous algae. Xiong and Sayre, Photosynthesis Research, 80, 411-419 (2004); Siripornadulsil et al., Microalgal vaccines. In: Transgenic Microalgae as Green Factories. Emilio Fernandes, Aurora Galvan, Rosa Leon, eds. Landes Press (2006). The invention is to be broadly understood as including methods of making the various embodiments of the recombinant algae cells of the invention described herein.

Transformation methods have also been developed for dinoflagellates (e.g., Ampidinium, Symbidinium), diatoms (e.g., Phaeodactylum, Cyclotella, Navicula, Cylindrotheca, Thalassiosira), green algae (e.g. Chlamydomonas, Chlorella, Haematococcus, Dunaliella), red algae (e.g., Kappaphycus), macroalgae (e.g., Ulva), Eustigmatophytes (e.g., Nannochloropsis; and bluegreen algae (e.g., Synechocystis, Synechococcus, Anabaena, Nostoc). The methods and protocols have been described in the literature and are incorporated here by reference. Chen et al., J Phycol 44: 768-776 (2008); Gokhale & Sayre, Photosystem II: Invited chapter in; Chlamydomonas in the Plant Sciences, volume 2, The Chlamydomonas Sourcebook, Elsevier 2008; Leon R, Fernandez D Advances in Experimental Medicine and Biology, Chapter 1, 616: 1-129 (2007); Packer L, Glazer A N, Meth Enzymol, 167: 1-91 (1988).

Strains of algae lacking cell walls are particularly useful for algal transformation, and are available for some of the aforementioned strains. The influence of the cell wall on delivery of molecules into and throughout the cell has been extensively studied in Chlamydomonas reinhardtii where a large number of cell wall free mutants are widely available. Azencott et al., Ultrasound Med Biol., 33, 1805-1817 (2007); Kindle et al., Proc Natl Acad Sci 88: 1721-1725 (1991) Additional cell wall free mutants are known for other algal genera such as Chlorococcales, Dunaliella and Chlorella. (Burczyk et al., Phytochemistry, 38, 837-841 (1995).

The nucleic acid fragment can be introduced into the cell using a vector, although “naked DNA” can also be used. The nucleic acid fragment can be circular or linear, single-stranded or double stranded, and can be DNA, RNA, PNA, or any modification or combination thereof The vector can be a plasmid, a viral vector or a cosmid. Selection of a vector or plasmid backbone depends upon a variety of desired characteristics in the resulting construct, such as a selection marker, plasmid reproduction rate, and the like. Suitable plasmids for expression in algae include, for example, Chlamydomonas nuclear transformation vectors e.g., pHyg3, pGenD, pSL18; Anacystis nidulans vectors pDF3, pUCC29, pSG11, and the like.

The expression vector can include one or more promoters that are operatively linked to a gene expressing a sugar metabolizing enzyme or a sugar transporter. The invention is not limited by the use of any particular promoter, and a wide variety of promoters are known. Promoters act as regulatory signals that bind RNA polymerase in a cell to initiate transcription of a downstream (3′ direction) coding sequence. The promoter used in the invention can be a constitutive or an inducible promoter. It can be, but need not be, heterologous with respect to the host cell. Examples of suitable promoters include the psaD promoter, the HSP70A promoter, and the actin promoter.

A promoter may be one that is naturally associated with a nucleic acid sequence. A naturally associated promoter may be obtained, for example, by isolating the 5′ non-coding sequences located upstream of the coding segment and/or exon. Such a promoter can be referred to as an “endogenous” promoter. Alternatively, certain advantages may be gained by positioning the coding nucleic acid segment under the control of a heterologous promoter, which refers to a promoter that is not normally associated with the specific nucleic acid sequence it is controlling in its natural environment. Such promoters may include promoters of other genes, and promoters isolated from any other virus, or prokaryotic or eukaryotic cell, and promoters or enhancers not “naturally occurring,” i.e., containing different elements of different transcriptional regulatory regions, and/or mutations that alter expression. Gene expression may be driven by a variety of heterologous promoters including those from Chlorella viruses as well as the CaMV 35S promoter (Mitra et al., Biochemical and Biophysical Research Communications 204: 187-194 (1994); Chen et al., Current Genetics 39, 365-370 (2001). Additional examples of suitable heterologous promoters include the SV40 promoter. the ubiquitin promoter, and the β tubulin promoter. The nucleic acid sequences of promoters can be prepared synthetically, or they can be produced using recombinant cloning and/or nucleic acid amplification technology, in connection with the compositions disclosed herein.

It may be preferable to provide specific types of promoters in the expression construction being used to prepare the recombinant oleaginous algae of the invention. For example, where the alga is of the Chlorella genus, the transforming plasmid could contain Chlorella-specific gene promoters to drive expression of the transgenes of interest. Likewise, if the alga is of the Dunaliella genus, the transforming plasmid could contain Dunaliella-specific gene promoters to drive expression of the transgenes of interest. A similar approach can be used for a wide variety of other types of algae, such as bluegreen algae and diatoms.

The nucleic acid fragment used to transform the alga cell can also include a termination sequence to end translation. A termination sequence is typically a codon for which there exists no corresponding aminoacetyl-tRNA, thus ending polypeptide synthesis. The nucleic acid fragment used to transform the host cell can optionally further include a transcription termination sequence. Examples of suitable terminators include rbcs2 terminators and actin terminators, as well as the psaD (e.g., from Chlamydomonas) terminator. A synthetic terminator sequence containing a 18 nt poly-G sequence can be used in place of the inverted repeat sequences found in typical transcriptional terminators. The poly-G sequence impedes the 3′ to 5′ exonuclease activity and stabilizes transcripts (Drager et al. RNA 2:652-663 (1996).

The control elements, such as a promoter and terminator, can be included in an operably linked manner to the genes expressing the sugar metabolizing enzymes and/or the sugar transport proteins to form an expression construct. For example, in one embodiment, the recombinant oleaginous alga includes a Chlorella kessleri HUP1 gene that is operatively linked to a Chlamydomonas psaD promoter and a Rbcs2 terminator.

The nucleic acid fragment used to transform the host cell optionally includes one or more marker sequences. The marker sequence can be used to select transformants on the basis of the phenotype imparted by the selectable marker gene. The marker sequence can encode a gene product, such as an enzyme, that inactivates or otherwise detects or is detected by a compound in the growth medium. For example, the inclusion of a selectable marker sequence can render the transformed cell resistant to an antibiotic, or it can confer compound-specific metabolism on the transformed cell. Examples of a selectable marker sequence are aadA, nptII, aphVII, aphVIII, etc., as well as sequences that confer resistance to kanamycin, ampicillin, chloramphenicol phleomycin, zeocin, neomycin, G418, paromomycin, hygromycin, erythromycin, polymyxin B sulfate, and tetracycline, or a marker designed to complement an ARG7 deletion mutant. Other possible selection schemes involve the use of genes which confer resistance to the inhibitors of carotenoid biosynthesis. These inhibitors include but are not limited to fluridone, norflurazone, and ketomorpholine A.

Sugar metabolizing enzymes and/or sugar transporter proteins can be expressed in the algal cell from an expression vector containing a nucleic acid fragment comprising the nucleotide sequence encoding the sugar metabolizing enzymes and/or sugar transporter proteins. Algae can be transformed either in the nucleus or in the chloroplast and different techniques apply depending on where the target insertion event is located. Integration of the nucleic acid fragment comprising the nucleotide sequence encoding the sugar metabolizing enzymes and/or sugar transporter is generally random in the nucleus, While this is a relatively low efficiency process, transformation occurs at a predictable rate and results in a stable construct in the genome of the cell. A host cell, preferably E. coli, is transformed with a nucleic acid fragment comprising one or more genes expressing sugar metabolizing enzymes and/or sugar transporter proteins and the associated control elements, such that the one or more genes are amplified. The cloned genes can then be introduced into the oleaginous algae using a suitable technique such as particle gun mediated transformation. Transformants can be selected on the basis of the phenotype imparted by a selectable marker gene (e.g., an antibiotic resistance gene), or by the gene of interest itself (e.g., a gene that confers the ability to metabolize a sugar that the wildtype strain cannot).

With respect to chloroplast transformation, nucleic acid sequences, whether heterologous or endogenous with respect to the algae cell, can be introduced into an algal genome using homologous recombination. First, the gene of interest and a gene encoding a selectable marker are inserted into a plasmid that contains piece of DNA that is homologous to the region of the genome within which the gene of interest is to be inserted. Next this recombinagenic DNA is introduced into the alga, and clones are selected in which the DNA fragment containing the gene of interest and selectable marker has recombined into the chromosome at the desired location. The gene and drug resistant marker can be introduced into the alga via transformation as a linearized piece of DNA that has been prepared from any cloning vector. Clones are then verified using PCR and primers that amplify DNA across the region of insertion. PCR products from non-recombinant clones will be smaller in size and only contain the region of the chromosome where the insertion event was to take place, while PCR products from the recombinant clones will be larger in size and contain the region of the chromosome plus the inserted gene and selectable marker.

### Stacking Additional Genetic Modifications to Increase Production of Biofuel Precursors

In further aspects of the invention, the recombinant oleaginous alga can be transformed with additional genes to increase the production of biofuel precursors from natural saccharide carbon sources. These further modifications of the alga can be introduced using separate expression vectors, or they can be introduced as “stacked” genetic modifications on the same expression vector used to transform the alga with genes expressing sugar metabolizing enzymes or sugar transport proteins.

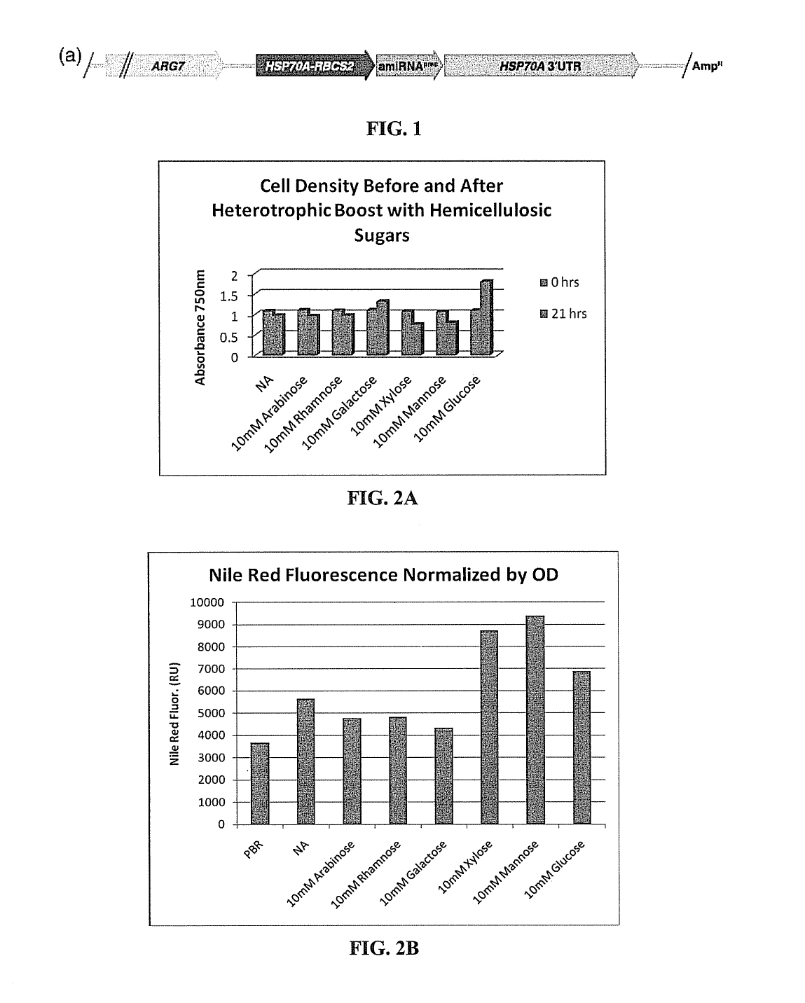

For stacking multiple gene constructs the transformation vectors are equivalent to those used for single gene inserts with the exception that unique gene promoters will be used to drive each unique gene cassette. These promoters include the psaD, ubiquitin, actin and rbcs promoters and their corresponding or heterologous 3′ ends or terminators. The transforming plasmid will also contain a selectable marker gene to identify transformants. An example of a three gene stack would include the hup sugar transporter, the AGPase miRNA construct, and a gene required for xylose metabolism, as shown in FIG. 1.

Additional modifications to the recombinant oleaginous alga can include 1) artificial RNAi constructs to inhibit the expression of ADP glucose pyrophosphorylase to block competing starch synthesis, 2) artificial RNAi constructs to inhibit expression of pyruvate decarboxylase to reduce pyruvate channeling to ethanol, 3) artificial RNAi constructs to inhibit expression of pyruvate formate lyase to reduce pyruvate channeling to formate, 4) overexpression of ACC synthase to increase fatty acid synthesis, and 5) overexpression of DGAT to increase triacylglycerol synthesis, and overexpression of caleosin and oleosin to increase oil storage capacity. These modifications may be stacked with one another to increase the production of biofuel precursors, and can also be stacked with any of the genes expressing sugar metabolizing enzymes or sugar transporting proteins.

The two major storage forms of reduced carbon in Chlorella are starch and oil. For example, in C. pyrenoidosa, starch accounts for 6% of the total biomass. However, starchless mutants of Chlorella have 22% higher growth rates than wild-type cells. Starchless cells also have the potential to produce more lipids than wild-type cells. When wild-type C. pyrenoidosa is nitrogen starved the lipid content increases from 15% to 25%. Under similar growth conditions the lipid content of starchless mutants increased to 38% of the total biomass, a 50% increase relative to wild-type cells. These results indicate that substantial gains in lipid content may be achieved by blocking starch synthesis in algae such as Chlorella.

Interfering RNA (RNAi) is part of a system within living cells that helps to control which genes are active and how active they are. RNAi includes both microRNA (miRNA) and small interfering RNA (siRNA). These small RNAs can bind to specific other RNAs and either increase or decrease their activity, for example by preventing a messenger RNA from producing a protein.

Accordingly, a further aspect of the invention provides a recombinant oleaginous alga using natural saccharides as a carbon source that further includes a nucleotide sequence expressing an RNAi construct complementary to all or a portion of the nucleotide sequence of a gene expressing ADP glucose pyrophosphorylase in order to decrease starch synthesis by the alga. To reduce fixed carbon sequestration as starch and increase oil accumulation, exemplary embodiments inhibit ADP glucopyrophosphorylase expression. AGPase catalyzes the first-dedicated and rate-limiting step in starch synthesis. Therefore, blocking AGPase activity will inhibit starch production. Specific embodiments introduce a Chlorella AGPase small subunit (catalytic subunit) RNAi hairpin element into the 3′ UTR (after the polyadenylation signal) of the terminator of the selectable marker gene (Zabawinski et al., J. Bacteriol., 183, 1069-1077 (2001)). To insure complete suppression of AGPase expression, one may also target RNAi elements for each member of the AGPase gene family. Enhanced accumulation of biofuel precursors in algae cells blocked for AGPase expression of up to 50% may result.

Additional unproductive uses of carbon by the recombinant oleaginous algae include the formation of ethanol and formate. The enzyme puruvate decarboxylase is part of the pathway leading to formation of ethanol from D-glyceraldehyde-3-phosphate that is formed from the sugar utilization pathway. Formate is also released during the conversion of this substrate to ethanol as a result of the activity of pyruvate formate lyase. Accordingly, RNAi constructs can be used to inhibit expression of pyruvate decarboxylase to reduce pyruvate channeling to ethanol and/or to inhibit expression of pyruvate formate lyase to reduce pyruvate channeling to formate.

Small interfering RNA and microRNA can be prepared using a number of methods. RNAi can be prepared enzymatically or by partial or total organic synthesis in which any modified ribonucleotide can be introduced by in vitro enzymatic or organic synthesis. Methods of synthesizing RNA molecules are known in the art, in particular, the chemical synthesis methods as described in Verma and Eckstein, Annul Rev. Biochem. 67:99-134 (1998). In another embodiment, a RNAi agent is prepared enzymatically. For example, a double stranded (ds)-siRNA can be prepared by enzymatic processing of a long dsRNA having sufficient complementarity to the desired target mRNA. Processing of long dsRNA can be accomplished in vitro, for example, using appropriate cellular lysates and ds-siRNAs can be subsequently purified by gel electrophoresis or gel filtration. The ds-siRNA can then be denatured according to art-recognized methodologies. The RNA can be purified from a mixture, if desired, by extraction with a solvent or resin, precipitation, electrophoresis, chromatography, or a combination thereof. Alternatively, the single-stranded RNAs can also be prepared by enzymatic transcription from synthetic DNA templates or from DNA plasmids isolated from recombinant bacteria. Typically, phage RNA polymerases are used such as T7, T3 or SP6 RNA polymerase (Milligan and Uhlenbeck, Methods Enzymol., 180, 51-62 (1989)). For a description of specific gene silencing by artificial RNAis in the unicellular alga Chlamydomonas reinhardtii, see Molnar et al., Plant J. 58:165-174 (2009).

The recombinant oleaginous algae of the invention includes one or more heterologous genes that increase the ability of the alga to use one or more natural saccharides for algal growth and can also contain one or more genes to increase lipid accumulation. These genes include: 1) the enzyme catalyzing the first-dedicated step in fatty acid synthesis, acetyl CoA carboxylase (ACCase) 2) the enzyme catalyzing triacylglycerol synthesis from diacylglycerol or diacylglycerol acyl transferase (DGAT); and 3) two genes encoding the functionally-related proteins; caleosin and olesin, which are required for the assembly of lipid storage vesicles in algae such as C. protothecoides (Frandsen et al., Physiologia Plantarum 112, 301-307 (2001); Hortensteiner et al., Plant Molecular Biology 42, 439-450 (2000)). For example, Chlorella caleosin expression levels increase dramatically when oil accumulation is induced.

Genes encoding ACCase and DGAT may be placed into transformation constructs and used to promote lipid accumulation in appropriate combination with the other genes of interest used. Overexpression of ACC synthase leads to increased fatty acid synthesis while overexpression of DGAT leads to increased triacylglycerol synthesis.

Overexpression of caleosin and oleosin leads to increased oil storage capacity in recombinant oleaginous algae. Generation of metabolite storage vesicles can facilitate the accumulation of biofuel precursors without an increase in the expression of the metabolite biosynthetic genes or the down-regulation of catabolic enzymes. The accumulation of hydrophobic compounds such as biofuel precursors may be influenced by the availability of protein-coated storage vesicles. To increase lipid accumulation in oil storage bodies, the genes encoding the lipid storage-body coat proteins, oleosin and caleosin, may be overexpressed in recombinant oleaginous algae. Elevated caleosin expression in C. protothecoides is correlated with enhanced lipid accumulation when cells are grown under conditions promoting lipid storage.

### Growth of Algae Culture and Extraction of Biofuel Precursors

The recombinant oleaginous alga of the invention can form a part of an algal culture. An algal culture refers to one or more algal species living in an environment that enables their survival and possible growth. The culture conditions required for various algae species are known to those skilled in the art. Examples of the components of an algal culture include water, carbon dioxide, minerals and light. However, the components of an algal culture can vary depending on the algal species, and whether or not conditions for autotrophic or heterotrophic growth are desired. For photoautotrophic growth, the algal culture will require CO₂ and light energy (e.g., sunlight), whereas heterotrophic growth requires organic substrates such as sugar for the growth of the algal culture, and can be carried out in the absence of light energy. As a result of being transformed to use natural saccharides as a carbon source, it is preferable to growth the recombinant oleaginous alga of the invention under conditions for heterotrophic growth. However, to encourage rapid growth of the algae before production of biofuel precursor, it may be preferable to grow the algae under autotrophic conditions prior to being placed under heterotrophic growth conditions. Additionally, mixotrophic growth (growth on fixed carbon in the presence of light) might also provide an advantage to specific algal strains. An algae culture requires that appropriate temperature conditions be maintained, and preferably that the culture is mixed to provide even access to nutrients and/or light. While some algae can grow in non-aqueous environments, algal culture as referred to herein is algal culture in an aqueous environment, and is therefore a liquid. Preferably the algal culture is a monoculture including a single algal species, or at least is intended as such, taking into account possible contaminating predators and competitors. Use of a monoculture makes it easier to provide optimal culture conditions, and can simplify growing and processing the algae in other ways. However, embodiments of the invention include algal cultures that have more than one species of algae present. The algal culture can be maintained in a pond, closed bioreactor, or other suitable site. While preparation of the recombinant oleaginous alga is carried out using relatively small cultures (e.g., 8 liter), production of biofuel precursors is preferably carried out using a larger culture, such as a 20,000 liter facility and much larger.

The major biofuel precursor produced by algae is oil or neutral lipids. Oils, including polar membrane lipids and triacylglycerols typically make up 5-15% of the total biomass of algae. Oils can accumulate, however, up to 60% (w/w) of the total biomass in many algae when they are grown under conditions where the capacity to produce or metabolize reduced carbon exceeds the energy and carbon demands for growth. Heterotrophic growth on reduced carbon enhances both cell division and sequestration of reduced carbon as oil, inducing facultative oil accumulation in the oleaginous algae.

Biofuel precursors, as used herein, refers to lipids and oils produced by algae that are organic compounds suitable for use in preparing a biofuel. While the lipids and oils will typically require additional processing before being used as biofuels, in some instances they may be used directly without additional processing. Lipids, as defined herein, include naturally occurring fats, waxes, sterols, monoglycerides, diglycerides, triglycerides, and phospholipids. The preferred lipids are fatty acid lipids found in triacylglycerides. Free fatty acids are synthesized in algae through a biochemical process involving various enzymes such as trans-enoyl-acyl carrier protein (ACP), 3-hydroxyacyl-ACP. 3-ketoacyl-ACP, and acyl-ACP. Examples of free fatty acids include fatty acids having a chain length from 14 to 20, with varying degrees of unsaturation. A variety of lipid-derived compounds can also be useful as biofuel and may be extracted from oleaginous algae. These include isoprenoids, straight chain alkanes (with short (3-7 carbon) and medium (8 to 12 carbon) chain lengths), and long and short chain alcohols, such short chain alcohols including ethanol, butanol, and isopropanol.

Biofuel precursors can be extracted from algal culture using any technique known to those skilled in the art. A preferred procedure is as described herein. Subsequent to increasing the lipid levels in the oleaginous alga, at least a portion of the algal culture is mixed with a lipid-extracting solvent to obtain a solvent-algae mixture, after which the mixture of the lipid-extracting solvent and the oleaginous algae is separated to obtain a solvent-lipid fraction and an extracted algae fraction. In some embodiments where the algae excrete biofuel precursors into the algal culture, the biofuel precursors can be extracted from the algal culture medium itself. However, in other embodiments the biofuel precursors are extracted from the algae themselves. Typically, only portions of the algal culture are removed to reduce the amount of lipid-extracting solvent required, to increase the effectiveness of extraction, and to minimize the additional stress on the oleaginous algae. The portions may be obtained in a continuous or non-continuous fashion, and in some embodiments the entire algal culture may be subjected to extraction. This extraction process is described in greater detail in U.S. Patent Publication No. 20090181438, which is incorporated herein by reference. To summarize, the cells are combined with a lipid-extracting solvent for a number of minutes in a process which has no significant effect on cell survivability. For example, the cells can be combined with a lipid extracting solvent for about 5 minutes. While the algae cells are in the lipid extracting solvent, it may also be preferable to briefly sonicate the algae cells (e.g., for a few seconds) at a frequency from about 20 kHz to 1 MHz, with frequencies of 20 KHz to 60 KHz being preferred.

Alternately, in some embodiments, it may be possible to extract the biofuel precursors from the medium of the algae culture without directly extracting the algae. In these embodiments, the biofuel precursors (e.g., short chain alkanes) are excreted into the medium for capture either directly or through recovery in the vapor phase.

A lipid-extracting solvent is an organic solvent that will take up lipids from oleaginous algae that are immersed in the solvent. Examples of lipid-extracting solvents include hydrocarbons with a length from C4 to C16, with hydrocarbons having a length of C10 to C16 being preferred. Examples of suitable lipid-extracting solvents include 1,12-dodecanedioic acid diethyl ether, n-hexane, n-heptane, n-octane, n-dodecane, dodecyl acetate, decane, dihexyl ether, isopar, 1-dodecanol, 1-octanol, butyoxyethoxyehteane, 3-octanone, cyclic paraffins, varsol, isoparaffins, branched alkanes, oleyl alcohol, dihecylether, and 2-dodecane. Oleaginous algae that have had their lipids removed by extraction are referred to herein as extracted algae.

Once the lipids have been extracted from the oleaginous algae, the lipid-extracting solvent and the oleaginous algae are separated to obtain a solvent-lipid fraction and an extracted algae fraction. This can be carried out in a phase-separation chamber, in which the aqueous solution including the extracted algae separates from the lipid-including lipid-extracting organic solvent. Because the solvents have different densities and affinities, they will naturally separate, with the aqueous solution typically forming a lower layer, while the organic solvent forms an upper layer. While this separation occurs naturally, it can be facilitated by applying sonication to decrease haze and emulsion formation. The solvent including the extracted lipid can then be collected from the phase separation chamber, and the solvent further processed (e.g., by distillation) to isolate and collect the lipid obtained.

The extracted algae fraction is typically then returned to the culture of oleaginous algae. The extracted algae fraction mixes back into the algal culture to form a part of that culture and is allowed to grow under non-stressed (e.g., nutrient replete) conditions, which allows the extracted algae to grow. Non-stressed refers to the normal culture conditions used for culturing the algae, in which the stress that was earlier applied is absent. An example of non-stressed conditions is nutrient replete conditions, in which all of the nutrients that are normally used by the algae are available in sufficient quantities that the algae are not subject to stress and will grow at their normal rate. Interestingly, extraction of the algae has been shown to extend culture growth times from one to more than five weeks under some conditions, which may be associated with the partitioning of waste products during the extraction. However, in the reverse situation, in which extraction has a somewhat detrimental effect on the algae, it may be preferable to carry out the extraction on only a fraction (e.g., 10-50%) of the overall algal culture in order to provide the algal culture with an opportunity to rebound and replace damaged cells during the non-stress period. Preferably, the oleaginous algae are allowed to grown under non-stressed conditions for about 12 to about 48 hours, though in other embodiments the algae may be allowed to grow for from 24 to 72 hours, or from 6 to 24 hours.

Examples have been included to more clearly describe particular embodiments of the invention. However, there are a wide variety of other embodiments within the scope of the present invention, which should not be limited to the particular example provided herein.

### EXAMPLES

### Example 1

### Use of Alternative Carbon Sources to Support Growth and Oil Production of Chlorella protothecoides

The inventors have observed that the green alga, Chlorella protothecoides, will substantially increase both its biomass and percent oil content when grown photoheterotrophically or heterotrophically with various sugars. In the presence of 10 mM glucose, Chlorella protothecoides oil yields increased by as much as 50 fold compared to yields obtained when grown autotrophically.

A number of reduced carbon sources have been investigated for their ability to support growth and oil production in Chlorella protothecoides. The growth and oil yield responses of Chlorella protothecoides to various hemicellulosic sugars (10 mM for 21 hours) is shown in FIG. 2. Providing reduced carbon sources such as sugars or glycerol provides what is referred to as a “heterotrophic boost.” As can be seen from the data, maximum growth was achieved with glucose, with galactose also providing high growth. There was little growth induction achieved, however, with arabinose, rhamnose, xylose or mannose. In contrast to the growth results, the inventors observed increased oil accumulation, normalized for cell numbers, for xylose, mannose and glucose but no increase for galactose. These results indicate that different sugars have different potentials for oil and biomass production and that oil production and biomass yield are not necessarily be linked.

To determine the molecular basis for these observed differences in sugar metabolism, the inventors screened two recently sequenced Chlorella genomes (Coccomyxa species C-169; a.k.a. Chlorella vulgaris C169, and Chlorella NC64A) for the presence and absence of genes required for the uptake and metabolism of hemicellulosic sugars. As described by Stadler et al., the Chlorella HUP1-3 family of sugar-H⁺ symporters imports a diverse group of monosaccharides including; glucose, fructose, galactose, mannose and xylose when expressed in transgenic yeast. Stadler et al., Plant Physiol. 107, p. 33-4 (1995).

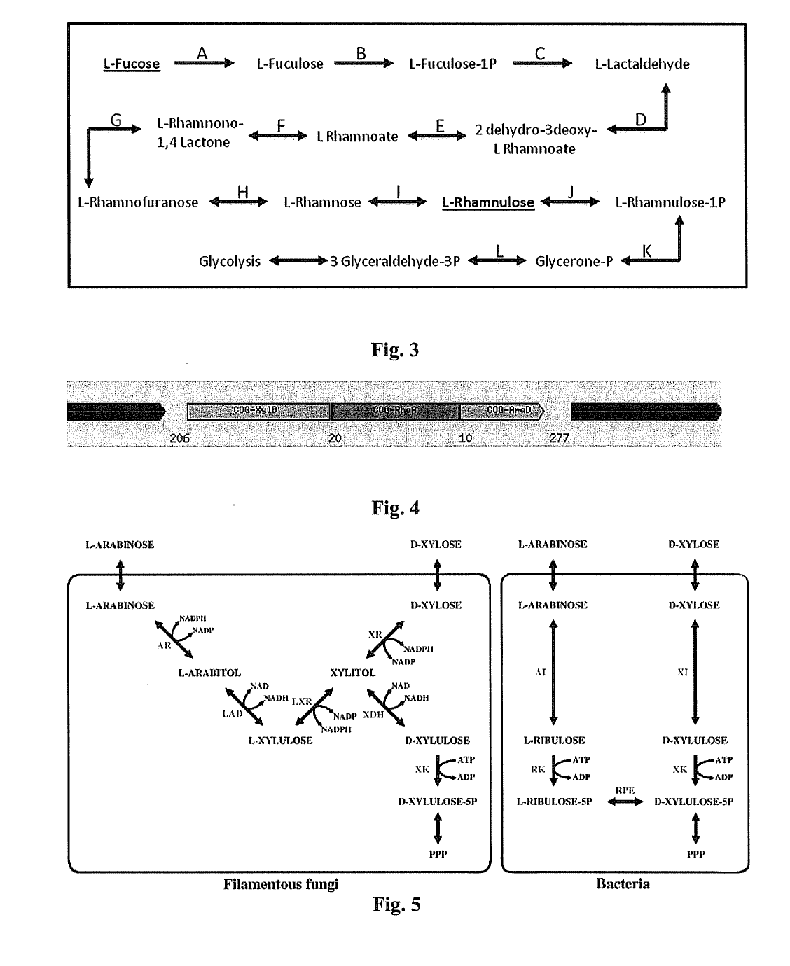

The ability to import hemicellulosic sugars is one aspect of sugar metabolism in algae. The genes and enzymes required for metabolizing each sugar are also important. The inventors have screened the two recently completed Chlorella genomes for the presence of genes involved in hemicellulosic sugar metabolism. FIG. 3 shows the enzymes involved in rhamnose metabolism, as represented by a letter between two sugar forms. Enzymes I through K, which are required for rhamnose metabolism, are typically missing from Chlorella genome.

TABLE 2
Candidate enzymes required for rhamnose metabolism in Chlorella
protothecoides.

| | | Gene | |
|---|---|---|---|
| | | present in | Gene present |
| | | Coccomyxa | in C. NC64A |
| Enzyme | Reaction | genome | genome |
| L-Rhamnose | L-Rhamnose → L- | No | No |
| isomerase (I) | rhamnulose | | |
| Rhamnulokinase (J) | ATP + L-rhamnulose | No | No |
| | → ADP + L- | | |
| | rhamnulose 1- | | |
| | phosphate | | |
| Rhamnulose-1- | L-Rhamnulose 1- | No | No |
| phosphate aldolase | phosphate → | | |
| (K) | glycerone phosphate + | | |
| | (S)-lactaldehyde | | |
| Triose-phosphate | D-Glyceraldehyde 3- | Yes | Yes |
| isomerase (L) | phosphate → | | |
| | glycerone phosphate | | |

The enzymes involved in rhamnose metabolism in Chlorella protothecoides are shown in Table 2. After transport, the metabolism of rhamnose is achieved through an isomerase, kinase and aldolase. Enzymes for the metabolism of fucose and rhamnose are not common among eukaryotic organisms. However, a wide range of prokaryotic organisms are capable of metabolizing these sugars including Clostridium sp., Bacillus subtilis, Yersinia pestis, and Escherichia coli. Moralejo et al., J Bacteriol. 175, p. 5585-94 (1993). The genes encoding the proteins responsible for metabolizing rhamnose in Clostridium belong to a single three gene rhaABD operon which can be cloned and expressed in Chlorella, as shown in FIG. 4.

Additionally, Galdieria sulphuraria, a thermoacidophilic red alga is capable of utilizing 27 different substrates including rhamnose and fucose. Gross et al., Plant Physiol. 114(1), pg. 231-236 (1997). In the case where the Clostridial operon is not functional in Chlorella, the pathways for rhamnose metabolism can be cloned from Galdieria. However, as Galdieria is an extremophile the codon usage and % G+C content of the genome may be optimized for growth under high temperature and low pH. To facilitate expression a functional protein from Galdieria, it may be preferable to alter the codon usage to mimic that of Chlorella. This has been done routinely in other organisms with known genomes and can be achieved at the molecular level after cloning the relevant genes in E. coli.

TABLE 3
Candidate genes/enzymes required for arabinose metabolism in Chlorella
protothecoides.

| | | Gene | Gene |
|---|---|---|---|
| | | present in | present in |
| | | Coccomyxa | C. NC64A |
| Enzyme | Reaction | genome | genome |
| L-arabinose 1- | L-arabinose + NAD+ → | No | No |
| dehydrogenase | L-arabinono-1,4- | | |
| | lactone + NADH + H+ | | |
| L- | L-arabinono-1,4- | No | No |
| arabinonolactonase | lactone + H₂O → | | |
| | L-arabinonate | | |
| L-Arabinonate | L-Arabinonate → 2- | No | No |
| dehydratase | dehydro-3-deoxy-L- | | |
| | arabinonate + H2O | | |
| oxidoreductase | 2-Dehydro-3-deoxy-D- | No | No |
| | xylonate + NAD+ → 5- | | |
| | Hydroxy-2,4- | | |
| | dioxopentanoate + | | |
| | NADH | | |
| Hydrolase | Glycolate + | No | No |
| | Pyruvate → 5-Hydroxy- | | |
| | 2,4-dioxopentanoate + | | |
| | H₂O | | |

Multiple pathways exist for incorporating arabinose into biomass from a variety of organisms. The pathways in FIG. 5 show the initial steps of pentose metabolism in filamentous fungi and bacteria, which lead to the pentose phosphate pathway and glycolysis. The following abbreviations are included in the Figure: XI, d-xylose isomerase; XK, d-xylulose kinase; AI, 1-arabinose isomerase; RK, 1-ribulokinase; RPE, 1-ribulose-5-phosphate 4-epimerase;)(a, d-xylose reductase; XDH, xylitol dehydrogenase; AR, 1-arabinose reductase; LAD, 1-arabitol 4-dehydrogenase; and LXR, 1-xylulose reductase. Recent work on strains of Pichia sp., a yeast known for fast conversion of xylose to ethanol suggest a similar pathway should be found in the sequenced genome of Pichia stipitis. Fonseca et al., FEBS J. 274, pgs. 3589-600 (2007). However, as of yet, the corresponding genes have not been annotated as such. Genes encoding enzymes for the prokaryotic pathway can be found in Klebsiella pneumonia and E. coli. An additional source of genes encoding arabinose metabolism is G. sulphuraria. Gross et al. identified a NADH dependent reductase with specificity for pentose sugars which is likely to be the first step in arabinose utilization after transport. Gross et al, Plant Physiol. 114, pgs. 231-236 (2007).

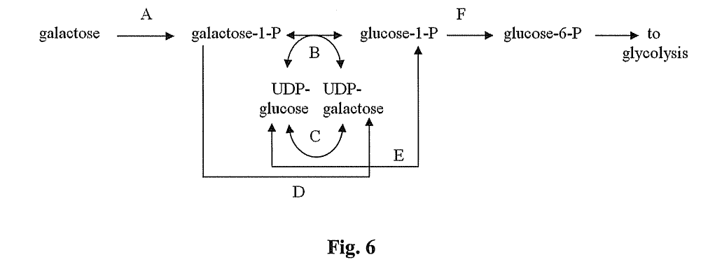

FIG. 6 illustrates a pathway for metabolizing free galactose in a cell. The first step in metabolizing free galactose involves phosphorylation via galactokinase (enzyme A) to form galactose-1-P. The ability to use galactose as a carbon source depends on whether an organism has the enzymes to further metabolize galactose-1-P. Chlorella NC64A and Coccomyxa C-169 each have one annotated copy of galactokinase in their respective genomes, but appear to lack the uridyltransferase enzymes (B and D in FIG. 6) to further metabolize galactose-1-P to glucose-1-P. UDPglucose-hexose-1-phosphate uridylyltransferase (enzyme B) would be the natural target for genetic engineering. Of the sequenced algal genomes, only the red algal extremophile Galdieria sulphuraria has an annotated copy of this gene. Alternative sources are the yeasts Saccharomyces cerevisiae and Trichoderma reesei. For this to be effective, C. protothecoides should have a copy of UDP-glucose 4-epimerase (enzyme C), to avoid accumulation of UDP-galactose. While annotated copies of this gene can be found in most of the sequenced algal genomes, including two copies in Chlorella NC64A, there is no annotated copy in Coccomyxa C-169. If the epimerase cannot be identified in C. protothecoides, the Chlorella NC64A version of the epimerase could be engineered.

TABLE 4
Enzymes involved in the metabolism of galactose

| Enzyme | Reaction |
|---|---|
| Galactokinase (A) | galactose → galactose-1-P |
| UDP glucose-hexose-1- | galactose-1-P + UDP-glucose [symbol] |
| phosphate uridylyltransferase | UDP-galactose + glucose-1-P |
| (B) | |
| UDP-glucose 4-epimerase (C) | UDP-glucose [symbol] UDP-galactose |
| UTP-hexose-1-phosphate | UTP + galactose-1-P [symbol] UDP- |
| uridylyltransferase (D) | galactose + PPᵢ |
| UTP-glucose-1-phosphate | UTP + glucose-1-P [symbol] UDP- |
| uridylyltransferase (E) | glucose + PPᵢ |
| Phosphoglucomutase (F) | glucose-1-P [symbol] glucose-6-P |

Chlorella protothecoides may already have the endogenous transporter system required to take up free galactose into the cell. The multi-sugar HUP transport system has been identified in the related species Chlorella kessleri. Sauer N, and Tanner W., FEBS Letters, 259, pg. 43-46 (1989). Specifically, the HUP2 protein has a demonstrated ability to transport galactose but has a low affinity for glucose. The Arabidopsis multi-sugar transporter STP1 can transport galactose as well as other sugars found in hemicelluloses, and would be a good alternative candidate for genetic engineering galactose transport. Boorer et al., J. Biol. Chem., 269, pgs. 20417-20424 (1994)

With respect to mannose metabolism, there are two ways that mannose can be metabolized once it is in the cell. The pathway used by eukaryotes converts D-mannose to D-fructose-6-P in two steps, as shown in Scheme 1 below.

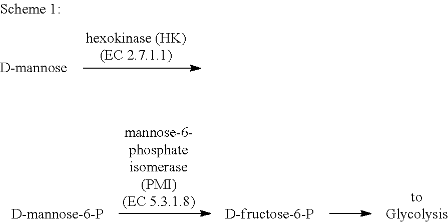

Chlorella protothecoides may not require genetic engineering in order to utilize mannose as a carbon source. Chlorella NC64A and Coccomyxa C-169 have annotated copies of both HK and PMI in their respective genomes. Substrate specificity may be an issue, since most hexokinases phosphorylate multiple substrates, with the highest affinity for glucose and fructose. There are reportedly some HKs that have as strong of an affinity for mannose as for glucose, e.g., human hexokinase I and Drosophila Hex A and Hex B.

Chlorella protothecoides has the ability to take up free mannose based on the observed increase in oil production with mannose. Importantly, the Chlorella kessleri HUP1, HUP2 and HUP3 transporters have been shown to transport mannose, although at a rate 32-55% of the rate for glucose. Stadler et al., Plant Physiol. 107, p. 33-4 (1995). Overexpression of the HUP transporters could be useful if mannose uptake is rate-limiting. In addition, the Arabidopsis STP1 multi-sugar transporter may be an alternative candidate for genetic engineering, since it has a demonstrated ability to transport mannose, galactose, xylose, fucose, and arabinose.

There are two general pathways for xylose metabolism, the xylose reductase (XR)-xylitol dehydrogenase (XDH) pathway, and the xylose isomerase pathway. The former occurs in eukaryotes (mainly yeasts) and the latter in bacteria.

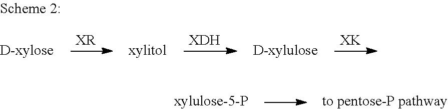

TABLE 5
Enzymes involved in the metabolism of xylose

| Enzyme | Reaction |
|---|---|
| Xylose reductase (XR) | D-xylose → xylitol |
| Xylitol dehydrogenase (XDH) | xylitol → D-xylulose |
| Xylulokinase (XK) | D-xylulose → xylulose-5-P |

Hawkins has reported on the ability of a Chlorella-like alga to grow on xylose after repeated mutagenesis by UV light and selection on medium with xylose as the only carbon source. Hawkins, R., Current Microbiology, 38, 360-363 (1999). Based on gene annotations, Chlorella NC64A and Coccomyxa C-169 would not be expected to metabolize xylose due to the absence of XR (NC64A) and XDH (both strains). However, the inventors observed substantial increases in oil production in C. protothecoides when grown on xylose, suggesting that C. protothecoides has a complete complement of genes necessary for xylose metabolism.

These results demonstrate that a number of different genes are useful candidates for transfection into oleaginous algae, including those involved in the metabolism of hemicellulosic sugars (rhamnose (3), arabinose (5) and galactose (2)) which are not metabolized by the wild-type form of Chlorella protothecoides, as shown in Table 6.

TABLE 6
Summary of possible number of genes required for hemicellulosic sugar
metabolism in Chlorella protothecoides.

| | Number of | Chlorella |
|---|---|---|
| | genes missing from | protothecoides sugar |
| Sugar | Chlorella genome | metabolism |
| Rhamnose | 3 | No |
| Arabinose | 5 | No |
| Galactose | 2 | Maybe |
| Mannose | 0 | Yes |
| Xylose | 0-1 | Yes |

Engineering Hemicellulosic Sugar Metabolism in Chlorella protothecoides.

There are three hemicellulosic sugars, rhamnose, arabinose and galactose, which are apparently not utilized by C. protothecoides for oil production. With the possible exception of rhamnose, the broad specificity sugar/proton symporters necessary for the import of each of these hemicellulosic sugars are present in Chlorella and assumed to be sufficient. C-14 labeled hemicellulosic sugar uptake experiments similar to those described by Stadler et al. can be carried out to confirm this. Stadler et al., Plant Physiol. 107, p. 33-4 (1995). If any of the hemicellulosic sugars are not transported, C. protothecoides can be transformed with the Arabidopsis multi-sugar transporter STP1 to increase the range of sugars transported.

To successfully express transgenes in C. protothecoides a robust genetic transformation system is required as well as the availability of transformation vectors containing a variety of strong gene promoters. The inventors have been able to transform C. protothecoides routinely and with high efficiency by particle gun bombardment using vectors containing antibiotic resistance genes as selectable markers. They have been able to use Chlamydomonas reinhardtii gene promoters to drive transgene expression in C. protothecoides. The ability to use well-characterized heterologous promoters from Chlamydomonas can greatly increase the range of transgene expression patterns possible.

After identification and verification (PCR, RT-PCR and DNA sequence analysis) of at least five independent transgenic algal lines for each transgene, recombinant algae can be grown under heterotrophic (dark) and photoheterotrophic growth conditions with and without the appropriate sugar (10 mM) and growth rates compared to non-transgenic algae grown in the presence and absence of sugar. The oil content of these cells can be readily compared to wild-type cells using the Nile red oil assay.

If a single gene (assumed to be required for metabolism of a specific sugar) is insufficient to allow sugar-dependent growth and oil accumulation, multiple vector constructs containing the full complement of genes assumed to be required for metabolism of the sugar of interest can be used to transform the algal cells.

### Example 2

### Survey of Wild Algal Species for Heterotrophic Growth and Oil Production on Hemicellulosic Sugars

In addition to engineering C. protothecoides, the inventors can also identify algae naturally capable of metabolizing a broad range of hemicellulosic sugars. These algae may be directly useful, or may be useful candidates for additional genetic modification. Multiple locations can be sampled for hemicellulosic metabolizing algae. For example, a survey can be carried out across a longitudinal transect stretching from Thunder Bay, Ontario, located on Lake Superior to Port St. Joe, Fla., located on the Gulf Coast for microalgae capable of enhanced oil accumulation and heterotrophic growth on hemicellulosic sugars. The hemicellulosic sugars tested can include; glucose, xylose, mannose, galactose, rhamnose and arabinose. Algae will be sampled from tree bark and pulp mill effluent ponds. These three sites vary in their forest species composition and climate. The dominant hemicellulosic sugars vary between the different tree species (e.g., deciduous and conifer) growing at these separate sites.

The locations of collected algal samples can be noted by GPS. The algae are initially grown photosynthetically on four different nutrient media including, Chlamydomonas high salt media, F2 media, Bold's media and proteose media. Algal samples will then be serially washed, diluted and plated on petri dishes containing autotrophic growth media plus ampicillin and fungicides to reduce contamination and to obtain single colonies. Cultures are maintained at high light until returned to the lab where single colonies are examined microscopically for contamination. The algae are subsequently purified of biological contaminants using standard procedures including brief sonication, detergents, antibiotics and differential centrifugation. Axenic cultures will be maintained on single plates. To track all steps in the process and to maintain records of all treatments, a bar-coding labeler and reader can be used so that all manipulations are electronically recorded. To avoid possible cross-contamination of cultures, only one isolate should be handled at a time in sterile transfer hoods before moving to a different sample. At number (e.g., 100) of independent single colony isolates can be obtained from each site.

To determine their relative growth rates and oil accumulation on various hemicellulosic sugars, cells are grown heterotrophically in liquid culture at 25° C. in minimal media supplemented with each separate sugar (10 mM) plus atrazine (20 μM) to block photosynthetic growth. Colony isolates capable of growing on a hemicellulosic sugars are then be grown photosynthetically and mixotrophically (light plus 10 mM sugar) to determine relative growth rates (light scattering at 750 nm), biomass (dry weight) and oil accumulation rates using a standardized Nile red assay. Cell lines exhibiting growth on hemicellulosic sugars can then be grown over a range of sugar concentrations to determine the optimal sugar concentration for growth and oil accumulation.

The identity of each strain can be determined by DNA sequence analysis of the 16S and 18S rRNA. Phylogenetic relationships are determined using standard boot-strap analysis programs. This will also determined whether algae having high oil production yields when grown on hemicellulosic sugars can be stored frozen. The ability to cryopreserve algae is useful for preventing the accumulation of mutations over time in lab strains. A standard two-stage freezing procedure can be used with various cryo-protectants to store the algae at liquid nitrogen temperatures.

The complete disclosure of all patents, patent applications, and publications, and electronically available material cited herein are incorporated by reference. However, should there be any conflict between material incorporated by reference and the specification, the material in the specification controls. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A recombinant oleaginous alga comprising one or more heterologous genes that increase the ability of the alga to use one or more natural saccharides for algal growth.

2. The recombinant oleaginous alga of claim 1, wherein the natural saccharide is a cellulosic or hemicellulosic sugar or a monosaccharide derived therefrom.

3. The recombinant oleaginous alga of claim 1, wherein the one or more heterologous genes express one or more sugar metabolizing enzymes that are lacking in a wild-type form of the oleaginous alga.

4. The recombinant oleaginous alga of claim 3, wherein the natural saccharide is sucrose and the sugar metabolizing enzyme is an invertase.

5. The recombinant oleaginous alga of claim 3, wherein the sugar metabolizing enzymes are selected from the group of enzymes capable of metabolizing the saccharides glucose, rhamnose, arabinose, galactose, mannose, fucose and xylose.

6. The recombinant oleaginous alga of claim 5, wherein the saccharide is arabinose and the sugar metabolizing enzymes comprise one or more of 1-arabinose reductase, 1-arabitol 4-dehydrogenase, and 1-xylulose reductase.

7. The recombinant oleaginous alga of claim 5, wherein the saccharide is rhamnose and the sugar metabolizing enzymes comprise one or more of L-rhamnose isomerase, rhamnulokinase, and rhamnulose-1-phosphate aldolase.

8. The recombinant oleaginous alga of claim 5, wherein the saccharide is galactose and the sugar metabolizing enzymes comprise one or more of UDP glucose-hexose-1-phosphate uridyltranserase and UDP glucose-hexose-1-phosphate uridylyltranserase.

9. The recombinant oleaginous alga of claim 5, wherein the saccharide is xylose and the sugar metabolizing enzymes comprise one or more of xylose isomerase, xylose reductase, xylitol dehydrogenase, and xylulokinase.

10. The recombinant oleaginous alga of claim 5, wherein the saccharide is mannose and the sugar metabolizing enzymes comprise one or more of hexokinase, mannokinase, ATP-dependent fructokinase, and mannose-6-phosphate isomerase.

11. The recombinant oleaginous alga of claim 1, wherein the one or more heterologous genes express one or more sugar transporters that are lacking in a wild-type form of the oleaginous alga.

12. The recombinant oleaginous alga of claim 11, wherein the oleaginous alga further comprises one or more heterologous genes expressing one or more sugar metabolizing enzymes that are lacking in a wild-type form of the oleaginous alga.

13. The recombinant oleaginous algae of claim 11, wherein the sugar transporter is selected from the group consisting of STP1, HUP1, HUP2, and HUP3 transporters.

14. The recombinant oleaginous alga of claim 1, wherein the one or more heterologous genes comprise one or more promoters that are operatively linked to a gene expressing a sugar metabolizing enzyme or a sugar transporter.

15. The recombinant oleaginous alga of claim 14, wherein the promoter is apsaD promoter or an actin promoter.

16. The recombinant oleaginous alga of claim 1, wherein the alga is selected from the group consisting of Bacillariophyceae, Chlorophyceae, Cyanophyceae, Xanthophyceae, Chrysophyceae, Chlorella, Crypthecodinium, Schizocytrium, Nannochloropsis, Ulkenia, Dunaliella, Cyclotella, Navicula, Nitzschia, Cyclotella, Phaeodactylum, and Thraustochytrid genera.

17. The recombinant oleaginous alga of claim 16, wherein the alga is of the Chlorella genus.

18. The recombinant oleaginous alga of claim 17, wherein the alga is of the Chlorella protothecoides species.

19. The recombinant oleaginous alga of claim 17, wherein the heterologous genes comprise a Chlorella kessleri hup1 gene that is operatively linked to a Chlamydomonas psaD promoter and a rbcs2 terminator.

20. The recombinant oleaginous alga of claim 1, further comprising a nucleotide sequence expressing an RNAi construct complementary to all or a portion of the nucleotide sequence of a gene expressing ADP glucose pyrophosphorylase in order to decrease starch synthesis by the alga.

21. The recombinant oleaginous alga of claim 1, further comprising a nucleotide sequence expressing an RNAi construct complementary to all or a portion of the nucleotide sequence of a gene expressing pyruvate decarboxylase in order to decrease ethanol synthesis by the alga.

22. The recombinant oleaginous alga of claim 1, further comprising a nucleotide sequence expressing an RNAi construct complementary to all or a portion of the nucleotide sequence of a gene expressing pyruvate formate lyase to reduce formate synthesis by the alga.

23. The recombinant oleaginous alga of claim 1, further comprising a nucleotide sequence expressing an RNAi construct complementary to all or a portion of the nucleotide sequence of a gene expressing ACCase in order to increase fatty acid synthesis by the alga.

24. The recombinant oleaginous alga of claim 1, further comprising an expression vector for overexpressing DGAT in order to increase fatty acid synthesis by the alga.

25. The recombinant oleaginous alga of claim 1, further comprising an expression vector for overexpressing caleosin and/or oleosin in order to increase fatty acid accumulation within the alga.

26. The recombinant oleaginous alga of claim 1, further comprising an expression vector for overexpressing DGAT and caleosin or oleosin in order to increase fatty acid synthesis and accumulation in the alga.

27. A method of making biofuel precursors, comprising
growing an algal culture comprising a recombinant oleaginous alga of any one of the preceding claims using a natural saccharide as a primary carbon source, and extracting one or more biofuel precursors from the algal culture.

28. The method of making oil of claim 27, wherein the natural saccharide is a cellulosic or hemicellulosic sugar, or a monosaccharide derived therefrom.

29. The method of claim 27, wherein the biofuel precursors are extracted by mixing a portion of the algal culture with an oil-extracting solvent to obtain a solvent-algae mixture, partitioning the solvent-algae mixture to obtain a solvent-biofuel precursor fraction and viable extracted algae, and returning the viable extracted algae to the algal culture.
